(19) United States
(12) Patent Application Publication
(10) Pub. No.: US 2022/0175489 A1
(43) Pub. Date: Jun. 9, 2022

(54) IMPLANT SURGERY DRILL

(71) Applicant: DMAX CO., LTD., Daegu (KR)

(72) Inventor: Tae Seok PARK, Seoul (KR)

(21) Appl. No.: 17/284,910

(22) PCT Filed: Dec. 27, 2018
(86) PCT No.: PCT/KR2018/016796

§ 371 Date: Sep. 15, 2021

(30) Foreign Application Priority Data
Oct. 16, 2018 (KR) 10-2018-0123376

(51) Int. Cl. A61C 1/14; A61C 8/00; A61B 17/16

(52) U.S. Cl. CPC A61C 1/14; A61C 8/0089; A61B 17/1673; A61B 17/1637; A61B 17/1617

(57) ABSTRACT

An implant surgery drill capable of preventing accidents in advance by actively reinforcing coupling force between a cutting part for cutting the alveolar bone and a connecting part for connecting the cutting part to a handpiece is proposed. The drill can include a cutting part having a plurality of cutting blades that comprise saw teeth and are aligned in a circular shape so as to perforate the alveolar bone; and a connecting part interposed between the handpiece and the cutting part, wherein, specifically, the cutting part including: the saw teeth aligned in a circular shape at the upper end thereof; a cutting upper part extending downward from the saw teeth; and a cutting lower part protruding downward from the cutting upper part with an expanded diameter.

### TECHNICAL FIELD

The present invention relates to a surgery drill for dental implant capable of perforate grooves in an alveolar bone, specifically, to prevent accidents in advance by actively reinforcing coupling force between a cutting part for cutting the alveolar bone and a connecting part for connecting the cutting part to a handpiece.

### BACKGROUND ART

In general, implant surgery is a dental treatment technique in which a biocompatible implant body is implanted in positions where there is loss of extraction, so as to restore the function of the natural teeth. The method for implanting an implant body may be classified according to implant positions such as endosseous implant, submucosal implant, bone penetrating type implant, and endodontic implant, and recently, a method of using a handpiece after forming a perforation in an alveolar bone and coupling a thread-type implant is often used.

Meanwhile, since an implant type may be determined according to diameter, depth, and angle, the precise a drilling is emerging, as well as securing safety of drill without damaging other regions of the oral cavity of a treated patient.

Accordingly, a surgical drill for dental implant with enhanced functions such as usability, cleanliness, preciseness and safety is continuously developed, and as an example, a “surgical drill for implant” is disclosed in Korean Patent Application Publication No. 10-119662, and the above-mentioned prior art includes a cutting part for cutting the alveolar bone and a connecting part protruding downward from the bottom surface of the cutting part that is coupled with a handpiece, and in particular, it is a technique that the cutting part has a cylindrical shape with a diameter decreasing as towards the lower part and a plurality of saw teeth are arranged in a circularly to form a perforation with a minimal drilling.

In order to safely and precisely implant the patient's alveolar bone, the method is carried out by gradually expanding a perforation of small diameter while varying the size of the drill, and the above-mentioned prior art has to abide the inconvenience of separating both the connecting part and the cutting part from each other in the handpiece, furthermore, there has been a problem that all the connecting parts, which are configured integrally when the cutting part is replaced due to wear, have to be replaced.

To solve such a problem, there is provided a surgical drill for dental implant in which a cutting part and a connecting part are provided separately, and as an example, a “surgical drill for implant” is disclosed in Korean Patent Application Publication No. 10-1711613. This is a technique, in which a cutting part for forming a perforation in the alveolar bone, a connecting part for connecting the cutting part, and the handpiece are configured to be disassembled and coupled with each other, so that machinability is improved in manufacturing a drill as well as storing and maintenance after use is made easy. A mutual coupling of the cutting part and the connecting part as described above, a bolt part is provided on the bottom surface of the cutting part, and a nut is further provided on the upper surface of the connecting part for a rotational coupling.

On the other hand, as a method of forming a precise perforation in a patient's alveolar bone while varying the size of drill and expanding a perforation of the small diameter is performed, accordingly, a repetitive replacement is carried out from the cutting part to the connecting part.

Further, in general, a nut part is more likely to wear due to weaker durability thereof compared to a bolt part, wherein the durability of the connecting part is subjected to a serious wear while a plurality of cutting part are replaced and used in one connecting part. In addition, the cutting part also induces to replacement use for a certain period of time due to wear while cutting the alveolar bone and hygiene and maintenance problems due to the intricate configuration, and an economic burden on use has emerged.

Accordingly, there is a demand for a technique capable of minimizing economical burden by actively extending the replacement period as much as the connecting part even when the cutting part is used due to wear and hygiene problems caused by perforation process on the alveolar bone.

### DISCLOSURE OF INVENTION

### Technical Problem

Therefore, the present invention has been made to more actively solve the above-mentioned problems, and an object of the present invention is to provide a surgical drill for dental implant, which not only actively reinforcing coupling force between a cutting part and a connecting part, but also ensures durability and lifespan of the connecting part even the cutting part is replaced frequently.

### Technical Solution

To accomplish the above object, according to one aspect of the present invention, there is provided a surgical drill for dental implant comprising: a cutting part 10 having a plurality of cutting blades that comprise saw teeth and are aligned in a circular shape so as to perforate the alveolar bone; and a connecting part 20 interposed between the handpiece and the cutting part, wherein, specifically, the cutting part 10 including: the saw teeth 11 aligned in a circular shape at the upper end thereof; a cutting upper part 12 extending downward from the saw teeth; a cutting lower part 13 protruding downward from the cutting upper part with an expanded diameter; and a coupling hole 14 recessed from the bottom surface of the cutting lower part so as to guide the insertion of the connecting part, the connecting part 20 including: a coupling protrusion 21 inserted and interposed in the coupling hole of the cutting part; a stepped part 22 protruding downward from the bottom of the coupling protrusion with an expanded diameter, and constraining the insertion length of the coupling hole; and an extended part 23 extendedly protruding from the bottom surface of the stepped part so as to be connected to the handpiece, and the coupling protrusion 21 further has threads on the outer periphery thereof, and the coupling hole 14 further has threaded grooves corresponding to the threads of the coupling protrusion, so that the coupling protrusion and the coupling hole are fastened to each other by a bolt fastening method.

The bottom surface of the cutting lower part 13 includes an extending protrusion 15 having a smaller diameter protruding downwards compared to the outer periphery of cutting lower part; and a recessed part 24 corresponding to the extending protrusion that is further provided on the upper surface of the stepped part 22, thereby reinforcing coupling force according to an expansion of insertion depth between a coupling hole and a coupling protrusion, and a friction area between a protrusion part and a recessed part.

In addition, a plurality of latch protrusions 15a having a sloped surface at one side is provided at the lower end of the extending protrusion 15, and a plurality of holding protrusions 24a corresponding to the latch protrusions is further provided so as to prevent from undesirable separations by latching each other when fastened.

The outer periphery of the cutting lower part 13 may include a number marked according to the diameter of the cutting part.

A surgical drill for dental implant according to another embodiment of the present invention configured to include: a cutting part 10 having a plurality of cutting blades that include saw teeth and are aligned in a circular shape so as to perforate the alveolar bone; and a connecting part 20 interposed between the handpiece and the cutting part, wherein, the cutting part 10 including: the saw teeth 11 aligned in a circular shape at the upper end thereof; a cutting upper part 12 extending downward from the saw teeth; a cutting lower part 13 protruding downward from the cutting upper part with an expanded diameter; and a coupling part 16, in which at least one or more protruding downwardly from the bottom surface of the cutting lower part, the connecting part 20 including: a coupling protrusion 21 inserted to the internal space of the cutting part; a stepped part 22 protruding downward from the bottom of the coupling protrusion with an expanded diameter, and constraining the insertion length of the coupling hole; and an extended part 23 extendedly protruding from the bottom surface of the stepped part so as to be connected to the handpiece, and the coupling protrusion 21 further has threads on the outer periphery thereof, and the coupling hole 25 formed to pass therethrough in the same position as the coupling part 16, and a fixing nut 30 fastening to some exposed portions of the coupling part 16 on the bottom surface of a stepped part 22 by passing the coupling space 25.

### Advantageous Effects

According to the present invention including the configuration as described above, as the integration is achieved by fastening a screw thread between a coupling hole provided on the bottom surface of the cutting part and a coupling protrusion provided on the upper surface of the connecting part, the abrasion of the coupling protrusion due to assembling and disassembling in a repetitive replacement may be actively reduced to secure the durability and lifespan of the connecting part.

According to the present invention, latch protrusions and holding protrusions are further provided between a coupling hole and a coupling protrusion so that protection is given for decoupling due to a mutual cross-latching.

According to the present invention, the cutting part is formed by a combination of at least one coupling part protruding downward from the bottom surface of the cutting part and a coupling hole formed in the stepped part and a fixing nut that passes through the coupling hole and a portion of the coupling part exposed to the bottom surface of the stepped part. As the coupling force between the connecting parts is reinforced, and the precise perforation may stably performed, so that an advantage of creating a more comfortable implant environment is expected.

### BRIEF DESCRIPTION OF THE DRAWINGS

FIG. 1 is a perspective view of a surgical drill for dental implant according to an embodiment of the present invention.

FIG. 2 is an exploded perspective view of FIG. 1.

FIG. 3 is a perspective view of a surgical drill for dental implant according to another embodiment of the present invention.

FIG. 4 is a partially enlarged view of FIG. 3.

FIG. 5 is a perspective view of a surgical drill for dental implant according to an embodiment of the present invention.

FIG. 6 is a sectional view of FIG. 5.

### BEST MODE FOR CARRYING OUT THE INVENTION

Hereinafter, a configuration, operations, and effects of the present invention will be disclosed with reference to the attached drawings.

Objects, characteristics and advantages of the present invention will be more clearly understood from the detailed description as will be described below and the attached drawings. Before the present invention is disclosed and described, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. The corresponding parts in the embodiments of the present invention are indicated by corresponding reference numerals.

The present invention relates to a surgical drill for dental implant to perforate a groove in the alveolar bone.

According to the present invention, above all, there provided a surgical drill for dental implant that prevents accidents in advance by actively reinforcing coupling force between a cutting part for cutting the alveolar bone and a connecting part for connecting the cutting part to a handpiece.

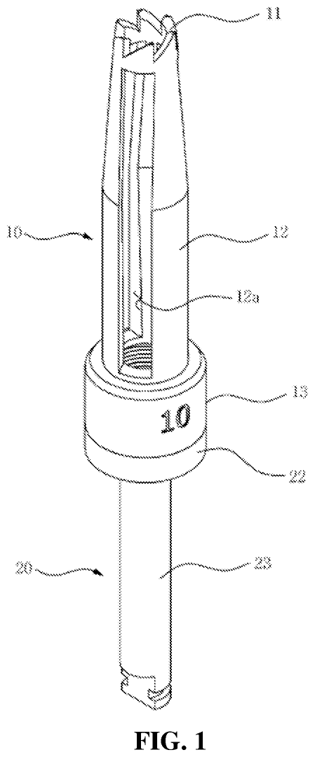

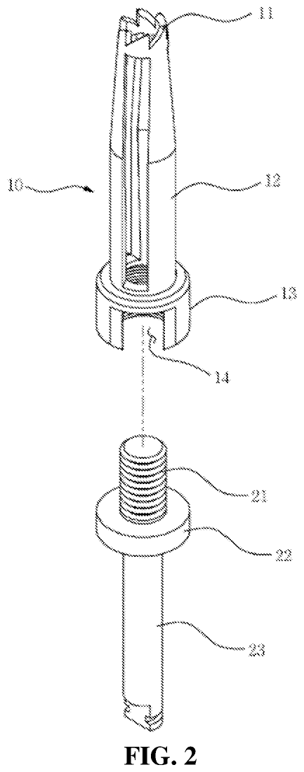

FIG. 1 is a perspective view perspective view of a surgical drill for dental implant according to a preferred embodiment of the present invention, and FIG. 2 is an exploded perspective view showing a combination relation between a coupling part perforating a groove in the alveolar bone and a connecting part connecting the handpiece.

As shown in FIG. 1 and FIG. 2, a surgical drill for dental implant of the present invention may be configured to include a cutting part 10 having a plurality of cutting blades that comprise saw teeth and are aligned in a circular shape so as to perforate the alveolar bone; and a connecting part 20 interposed between the handpiece and the cutting part.

The cutting part 10 may further include a cylindrical shape of cutting upper part 12 having a hollow part formed internally by communicating between top and bottom; saw teeth 11 having an alignment in a circular shape on the upper end thereof; a cutting lower part 13 protruding downward from the cutting upper part with an expanded diameter; and a coupling hole 14 recessed from the bottom surface of the cutting lower part so as to guide the insertion of the connecting part.

The saw teeth 11 may be aligned in a circular shape on the front end of the cylinder, and having a sloped surface at one side. A plurality of saw teeth 11 having the same configuration described above may cut a patient's alveolar bone in a circular shape while making a rotation about by a handpiece.

The cutting upper part 12 may be extended downward from the saw teeth and gradually expands the diameter from the center thereof. A tapered cutting upper part 12 having a gradual expansion of the diameter may guide to minimize the size replacement of the cutting part as the diameter gradually expand as the alveolar bone of the patient is cut, as well as precisely cut the alveolar bone.

In addition, the cutting upper part 12 further provided with a penetrating part 12a so as to communicate with an internal hollow part at a constant interval at one side, thereby friction heat generated between the cutting upper part and the alveolar bone may be dissipated to outside via the penetrating part and the hollow part, furthermore, fragments introduced from cutting the alveolar bone may be collected in the hollow part through the penetrating part.

The cutting lower part 13 may be protruding downward from the bottom with an more expanded diameter than the cutting upper part, and a space for guiding the coupling of connecting part 20, as described in greater detail below, may be provided, while providing a surface for assembling the cutting part so that a user can grip the outer periphery of the cutting lower part.

Meanwhile, on the outer periphery of cutting lower part including a number marked according to the diameter of the cutting part, therefore, a user may easily distinguish the size of the cutting part from the number marked.

The coupling hole 14 may be recessed towards the center from the bottom surface of the cutting lower part 13, and consequently, the coupling hole is formed to communicate with the internal space of the cutting upper part. Specifically, the coupling hole 14 further provided with a helical shaped screw thread in the internal circumference and guides a coupling of connecting part 20 as described in greater detail below.

The connecting part 20 is configured to include a coupling protrusion 21 inserted and interposed to the coupling hole of the cutting part, a stepped part 22 protruding downward from the bottom of the coupling protrusion with an expanded diameter, and constraining the insertion length of the coupling hole; and an extended part 23 extendedly protruding from the bottom surface of the stepped part so as to be connected to the handpiece.

The coupling protrusion 21 may be comprised in the length corresponding to the depth of the coupling hole 14, and a threaded groove is given so as to coupled with a helical shaped screw thread formed in the coupling hole correspondingly.

The stepped part 22 is formed such that protruded in a constant thickness with slightly expanded diameter compared to the coupling protrusion, wherein, the diameter of the stepped part is identical to the diameter of the cutting lower part, and may result fineness in terms of exterior of a surgical drill for dental implant.

The stepped part 22 may provide a gripping surface that guides the user's gripping like the cutting lower part 13 in the process of mounting the connecting part 20 and cutting part 10, and constrain the depth of the coupling protrusion 21 inserted to the coupling hole 14.

The extended part 23 at the end is fixedly coupled with a handpiece in the axis of protruding downwards from the bottom surface of the stepped part 22, and transfer the rotational power provided from the handpiece to the cutting part 10.

The cutting part 10 and the connecting part 20 including the configuration as described above may firmly fasten by rotation in the state of the coupling protrusion 21 disposed in the bottom surface of the coupling hole 14. By arranging the coupling hole 14 with slightly weak durability in the cutting part 10, and a coupling protrusion 21 with better durability compared to the coupling hole arranged to the connecting part 20, abrasion of the connecting part due to coupling and disassembling of the cutting part is prevented so as to improve the durability and lifespan of connecting part.

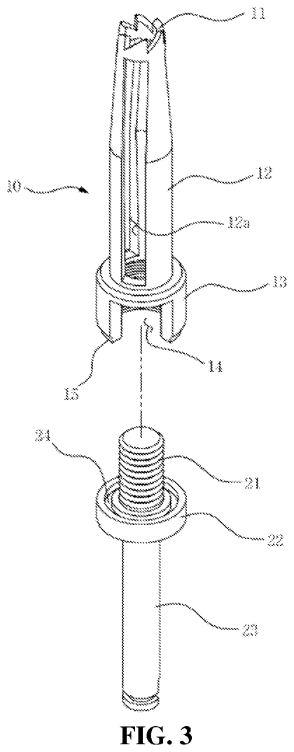

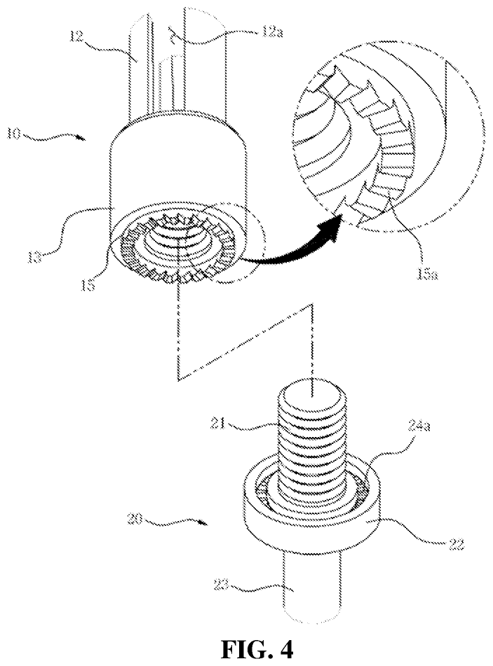

FIGS. 3 and 4 are perspective views illustrating a coupling relation of a cutting part and a connecting part configured according to another embodiment of the present invention.

As illustrated in FIG. 3, a surgical drill for dental implant of the present invention may further include an extending protrusion 15 having a smaller diameter protruding downwards compared to an outer periphery of the cutting lower part so as to expand the insertion depth; and a recessed part 24 corresponding to the extending protrusion on the upper surface.

For example, the extended protrusion 15 and a recessed part 24 are merely an example to help understand the present disclosure, and are not intended to limit the present invention. It should also be appreciated that the cutting lower part 13 and the stepped part 22 may be configured as various types of protrusion and recessed parts within the spatial range.

Meanwhile, as shown in FIG. 4, a surgical drill for dental implant of the present invention may further include a plurality of latch protrusion 15a having sloped surface at each one sides facing surfaces of extending protrusion 15 and a recessed part 24 respectively, and a plurality of holding protrusion 24a corresponding to the latch protrusion, so as to prevent from undesirable separations by latching each other when fastened.

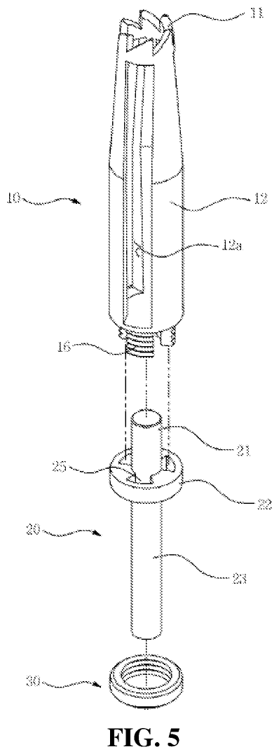

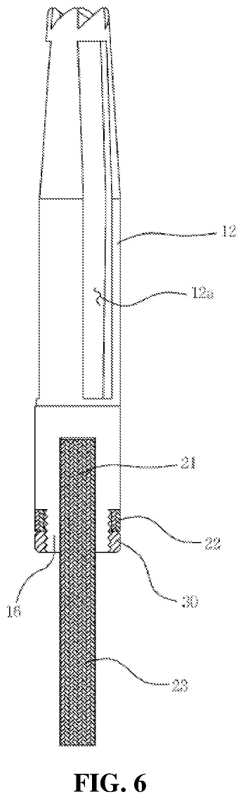

FIGS. 5 and 6 are perspective views are another embodiment of the present invention illustrating the reinforced coupling force of a surgical drill for dental implant by further implementing a fixing nut in the coupling relation between the cutting part and connecting part.

As illustrated in FIG. 5, a surgical drill for implant of the present invention may be configured to include a cutting part 10 having a plurality of cutting blades that comprise saw teeth and are aligned in a circular shape so as to perforate the alveolar bone; and a connecting part 20 interposed between the handpiece and the cutting part.

The cutting part 10 is configured to including the saw teeth 11 aligned in a circular shape at the upper end thereof; a cutting upper part 12 extending downward from the saw teeth; a cutting lower part 13 protruding downward from the cutting upper part with an expanded diameter; and a coupling part 16 at least one or more protruding downwardly from the bottom surface of the cutting lower part, the connecting part 20 including: a coupling protrusion 21 inserted to the internal space of the cutting part; a stepped part 22 protruding downward from the bottom of the coupling protrusion with an

expanded diameter, and constraining the insertion length of the coupling hole; and an extended part 23 extendedly protruding from the bottom surface of the stepped part so as to be connected to the handpiece, and the coupling protrusion 21 further has threads on the outer periphery thereof, and the coupling hole 25 included to pass therethrough in the same position as the coupling part 16, and a fixing nut 30 fastening to some exposed portions of the coupling part 16 on the bottom surface of a stepped part 22 by passing the coupling space 25.

As illustrated in FIG. 6, a plurality of coupling part protruded downwardly to the bottom surface of the cutting lower part 13 may enter into a coupling hole 25 formed in the same position as the coupling part on the stepped part, and some portions at the end may be arranged in the bottom surface of the stepped part exposed. A fixing nut 30 may be entered from the extended part 23 of connecting part 20, so as to the stepped part 22 may be rotationally coupled to the exposed coupling part 16, here, the cutting lower part 13 and a nut both apply pressure to the stepped part having a stepped part therebetween, resulting a firm fastening. In particular, a coupling hole 14 and a coupling protrusion 21 or a coupling part 16 and a fixing nut 30 of the present invention are both configured threaded groove and thread in opposite directions to the direction in which the handpiece rotates, so as to completely prevent undesired disassembly of the cutting part 10 on the connecting part 20 during the perforation process.

A surgical drill for dental implant according to the present invention including the configuration as described above, as the integration is achieved by fastening a screw thread between a coupling hole provided on the bottom surface of the cutting part and a coupling protrusion provided on the upper surface of the connecting part, the abrasion of the coupling protrusion due to assembling and disassembling in a repetitive replacement may be actively reduced to secure the durability and lifespan of the connecting part, and latch protrusions and holding protrusions are further provided between a coupling hole and a coupling protrusion so that protection is given for decoupling due to a mutual cross-latching. The cutting part is formed by a combination of at least one coupling part protruding downward from the bottom surface of the cutting part and a coupling hole formed in the stepped part and a fixing nut that passes through the coupling hole and a portion of the coupling part exposed to the bottom surface of the stepped part. As the coupling force between the connecting parts is reinforced, and the precise perforation may stably performed, so that a more comfortable implant environment is created.

Although the present invention has been described with reference to the embodiments shown in the drawings, this is only illustrative, and those skilled in the art may understand that various modifications and equivalent other embodiments are possible. The scope of the protection of the present application should be determined by reasonable interpretation of the appended claims and all technical concepts coming within the equivalence range of the present invention should be interpreted to be embraced in the scope of the right of the present invention.

### DESCRIPTION OF SYMBOLS

| | |
|---|---|
| 10: Cutting part | 11: Saw teeth |
| 12: Cutting upper part | 13: Cutting lower part |
| 14: Coupling hole | 15: Extending protrusion |
| 16: Coupling part | 20: Connecting part |
| 21: Coupling protrusion | 22: Stepped part |
| 23: Extended part | 24: Recessed part |
| 25: Coupling space | 30: Fixing nut |

1. A surgical drill for dental implant comprising:
a cutting part having a plurality of cutting blades that comprise saw teeth and are aligned in a circular shape so as to perforate the alveolar bone; and a connecting part interposed between the handpiece and the cutting part, wherein the cutting part 10 includes:
the saw teeth 11 aligned in a circular shape at the upper end thereof; a cutting upper part 12 extending downward from the saw teeth; a cutting lower part 13 protruding downward from the cutting upper part with an expanded diameter; and a coupling part 16 at least one or more protruding downwardly from the bottom surface of the cutting lower part,
the connecting part includes:
a coupling protrusion inserted and interposed to the coupling hole of the cutting part, a stepped part protruding downward from the bottom of the coupling protrusion with an expanded diameter, and constraining the insertion length of the coupling hole; and an extended part extendedly protruding from the bottom surface of the stepped part so as to be connected to the handpiece, and
the coupling hole further provided with a threaded groove corresponding to a thread of the coupling protrusion, so that the coupling protrusion and the coupling hole are fastened to each other by a bolt fastening method.

2. A surgical drill according to claim 1, wherein the bottom surface of the cutting lower part comprising:
an extending protrusion 15 having a smaller diameter protruding downwards compared to the outer periphery of cutting lower part; and
a recessed part corresponding to the extending protrusion that is further provided on the upper surface of the stepped part, thereby reinforcing coupling force according to an expansion of insertion depth between a coupling hole and a coupling protrusion, and a friction area between a protrusion part and a recessed part.

3. The surgical drill according to claim 2, wherein a plurality of latch protrusions having a sloped surface at one side is provided at the lower end of the extending protrusion, and a plurality of holding protrusions corresponding to the latch protrusions is further provided so as to prevent from undesirable separations by latching each other when fastened.

4. The surgical drill according to claim 1, wherein the outer periphery of the cutting lower part comprising a number marked according to the diameter of the cutting part.

5. A surgical drill for dental implant comprising:
a cutting part having a plurality of cutting blades that comprise saw teeth and are aligned in a circular shape so as to perforate the alveolar bone; and a connecting part interposed between the handpiece and the cutting part,
the saw teeth aligned in a circular shape at the upper end thereof; a cutting upper part extending downward from the saw teeth; a cutting lower part protruding downward from the cutting upper part with an expanded diameter; and a coupling part at least one or more protruding downwardly from the bottom surface of the cutting lower part,
the connecting part is configured to include a coupling protrusion inserted into the internal space of the cutting part, a stepped part protruding downward from the bottom of the coupling protrusion with an expanded diameter, and constraining the insertion length of the coupling hole; and an extended part extendedly protruding from the bottom surface of the stepped part so as to be connected to the handpiece, the coupling hole included to pass therethrough in the same position as the coupling part, and
a fixing nut fastening to some exposed portions of the coupling part on the bottom surface of a stepped part by passing the coupling space.
